(19) United States
(12) Patent Application Publication
(10) Pub. No.: US 2005/0271949 A1
(43) Pub. Date: Dec. 8, 2005

(54) Reticle manipulations

(76) Inventors: Scott Corboy, Singapore (SG); Ronald Roes, Nijmegen (NL); Hennie De Weerd, Siebengewald (NL)

Correspondence Address: CHRISTIE, PARKER & HALE, LLP, PO BOX 7068, PASADENA, CA, 91109-7068, US

(21) Appl. No.: 10/524,280

(22) PCT Filed: Dec. 9, 2002
(86) PCT No.: PCT/SG02/00287

§ 371 Date: Feb. 8, 2005

(51) Int. Cl. G21G 5/00; A61N 5/00; H01J 37/08; G03F 1/00; G21K 5/10

(52) U.S. Cl. 430/5; 250/492.2; 250/492.22

(57) ABSTRACT

Slits and holes within a reticle for a local interconnect layer (LIL) are sized according to factors such as relative isolation and whether they are large or small. This helps overcome side-lobing and other undesirable effects of interference.

### FIELD OF THE INVENTION

The present invention relates to reticle manipulation to design masks for use in photolithography, and in particular it relates to such masks for use in deep-submicron integrated circuit technologies and more particularly for producing the local interconnect layer (LIL).

### BACKGROUND ART

Lithography is used in the production of integrated circuits to aid in material additions to and removals from a silicon substrate. Typically light or electron beams (or another controlled source of energy) of a particular wavelength passes through a pattern on a mask onto the substrate which has been treated with a chemical resist. There they react with a resist to change its structure, allowing that altered resist to be dissolved away (or to allow the modified portions to be the only ones that do not dissolve away) as is desired. Modem masks have holes of very small sizes. As the sizes decrease, the problems associated with diffraction and constructive and destructive interference become more important and difficult to control.

The resolution of an exposure depends on the contrast between adjacent light and dark areas. The more light nominally dark areas receive, the lower the resolution with the higher the chance of adjacent discrete features being melded together. Replication quality depends largely on the amount of allowable exposure dose and depth of focus that still results in a correct image size.

The space saving Local Interconnect Layer (LIL), which is present in deep-submicron integrated circuit technologies, requires both small holes and slits. Examples of these are shown in FIG. 1, which is a top scanning electron microscope view of an SRAM after local interconnect layer photolithography. This SRAM was produced using a reticle designed as is discussed later and is discussed here not as prior art, but as being illustrative of what is required.

Such small features can be fabricated using Phase Shift Mask (PSM) technology. This approach varies the phase of an electric field vector by modifying the distance through which the lithographic beam travels through the mask. When beams of equal magnitude but which are 180 degrees out of phase meet, they cancel each other perfectly.

PSM lithography is used in the manufacture of memory chips. Currently, such small features usually involves the use of two masks, one for the holes and one for the slits. The LIL presents a considerable challenge to lithography since the different physics involved in exposing holes and slits makes mask sizing very complex. This problem is compounded by the use of Half-Tone Phase Shift Mask (HT-PSM) reticles, which are necessary for producing the current small sizes, which makes correct sizing even more complex. In HT-PSM the reticle is covered in a partially transparent material such as molybdenum silicide (MoSi). Unlike chrome, MoSi allows a small percentage of the light to pass through (typically 6% or 18%). By choosing an appropriate thickness of the MoSi the light that does pass through can end up 180° out of phase with the light that passes through the neighbouring clear glass areas. This light that passes through the MoSi areas is too weak to expose the resist but it does interfere with the light passing through the clear areas, with the phase difference resulting in destructive interference. This allows sharper and, therefore, smaller features to be printed on the wafer. Whereas other types of PSM, such as alternating PSM can be directed to particular features, rather than the whole reticle (mask), Half-Tone PSM requires that the effect be applied to the complete reticle, rather than just parts or it.

Patent document U.S. Pat. No. 5,807,649 describes a lithographic patterning method which uses two masks. Exposure first occurs using a first, edge phase shifted mask. A second exposure then occurs using a second, phase shift trim mask. The mask dimensions of opaque areas in the second mask have increased block sizes to remove previous exposure defects. In producing the trim mask, the entire protect mask design is enlarged by the worst case overlay error between the first and second masks. Additionally, protect shape features which are smaller than a minimum feature size are increased in size by one design grid per edge.

Patent document U.S. Pat. No. 6,316,163 describes a lithographic method in which a pattern is transferred onto a first layer of a material using both a light beam and an electron beam, using information that is also to be transferred onto a second layer. Initial oversizing of patterns occurs, followed by subtractions of patterns or other logical functions to avoid overlapping areas between the patterns to be transferred to the two layers.

Patent document U.S. Pat. No. 6,255,024 describes a single phase shift mask system for use in 365 nm lithography. This system uses positive biasing, that is holes larger than the desired feature to compensate for destructive interference at the edges. This document in particular addresses the problem of side-lobes produced by interference between side-lobe light created at the edge of the pattern and main lobe sight transmitted by the attenuating material of the mask. This problem is addressed by including sub-resolution openings in the transmission areas with the attenuating material thereon.

Patent document U.S. Pat. No. 6,057,063 describes a system for converting a binary chip design into a phase-shifted mask layout. This includes selectively and repeatedly expanding portions of 180 degree phase shapes that are perpendicular to residual phase edges.

The trouble with using two masks is that it requires more material and time and adds to the complexity of production (not least in ensuring correct alignment between succeeding masks). The present invention aims to aid the design of masks that allow various features, such as small holes and slits to be exposed at the same time, in particular in the production of the LIL.

### SUMMARY OF THE INVENTION

According to one aspect of the present invention, there is provided a method for use in designing a reticle for exposing a substrate during production of a circuit, comprising:
determining the relative isolation of at least some of the features to be produced using said reticle; and
sizing the corresponding apertures in said reticle according to the determined relative isolation.

According to a second aspect of the present invention, there is provided a method for use in designing a reticle for exposing a substrate during production of a circuit, comprising:
determining a first dimension of at least some of the features to be produced using said reticle; and
sizing the corresponding apertures in said reticle by different amounts and proportions according to the determined first dimension.

According to a third aspect of the present invention, there is provided for use in designing a reticle for exposing a substrate during production of a circuit, comprising:
determining the relative isolation of at least some of the features to be produced using said reticle and sizing the corresponding apertures in said reticle according to the determined relative isolation; and
determining a first dimension of at least some of the features to be produced using said reticle and sizing the corresponding apertures in said reticle by different amounts and proportions according to the determined first dimension.

According to a fourth aspect of the present invention, there is provided a reticle for exposing a substrate during production of a circuit, comprising: a first class of apertures, whose sizes are dependent on the relative isolation of the features to be produced thereby.

According to a fifth aspect of the present invention, there is provided a reticle for exposing a substrate during production of a circuit, comprising: a second class of apertures, whose sizes are dependent on a first dimension of the features to be produced thereby, with said second class of apertures being sized with different relative and absolute amounts, relative to the sizes of the features to be produced, according to the size in a first dimension of the features to be produced.

According to a sixth aspect of the present invention, there is provided a reticle for exposing a substrate during production of a circuit, comprising:
a first class of apertures, whose sizes are dependent on the relative isolation of the features to be produced thereby; and
a second class of apertures, whose sizes are dependent on a first dimension of the features to be produced thereby, with said second class of apertures being sized with different relative and absolute amounts, relative to the sizes of the features to be produced, according to the size in a first dimension of the features to be produced.

Thus slits and holes within a reticle for a local interconnect layer (LIL) are sized according to factors such as relative isolation and whether they are large or small. This helps overcome sidelobing and other undesirable effects of interference.

### BRIEF DESCRIPTION OF THE DRAWINGS

The invention will now be further described by way of non-limitative example, with reference to the accompanying drawings, in which:

FIG. 1 is a top scanning electron microscope view of an SRAM after local interconnect layer photolithography using a first reticle;

FIG. 2 is a schematic view of a HT-PSM test reticle;

FIG. 3 is a more detailed view of a LIL test matrix of FIG. 3;

FIG. 4 is a table of hole and slit sizes used in the various reticles that were taped out in developing the present invention;

FIG. 5 is a top scanning electron microscope view of a controller portion of an SRAM after local interconnect layer photolithography using the first reticle;

FIG. 6 is a schematic view of a side-lobing weak point in a frequently appearing shape;

FIG. 7 is a graph showing the results for hole sizes produced by using a second reticle produced using a different set of rules;

FIG. 8 is a graph showing the results for slit widths produced by using the second reticle;

FIG. 9 is a graph showing the results for a constant slit length, produced by using the second reticle;

FIG. 10 is a graph showing the results for holes produced by using the second reticle, given their relative isolation;

FIG. 11 is a graph showing the results for short slits, produced by using the second reticle;

FIG. 12 is a top scanning electron microscope view of a controller portion of an SRAM after local interconnect layer photolithography using the second reticle;

FIG. 13 is a graph of hole size relative to the nearest feature, produced by using the second reticle;

FIGS. 14 and 15 are schematic diagrams for explaining the determination of the definition of an isolated hole;

FIG. 16 is a graph comparing the results for holes produced by using a third reticle, with the results from the second reticle;

FIG. 17 is a graph comparing the results for short slits produced by using the third reticle, with the results from the second reticle;

FIG. 18 is a top scanning electron microscope view of an SRAM after local interconnect layer photolithography using a reticle developed using the present invention;

FIG. 19 is a tilted view of the SRAM of FIG. 1 after some further processing; and

FIG. 20 is a flowchart for manipulating hole and slit sizes according to the invention.

### SPECIFIC DESCRIPTION

The present invention was derived following a series of experiments, main aspects of which are now described. In each instance, the aim was to produce an acceptable set of features following exposure using a single reticle solution for the LIL. Once a solution was obtained that was suitable across the board, it could be used to design and produce masks on an industrial basis. What was sought was a reticle or rules to produce a reticle that had a usable operating window of at least 0.6 μm (micron) depth of focus.

The reticles used are glass covered in a MoSi alloy. This is slightly transmittive (typically 6%) but at opposite (180 degree) phase. The reticles are exposed on a DUV scanner system (DUV=248 nm light), the DUV light being generated from a laser chamber.

The specific series of experiments were conducted using 180 nm CMOS Shrink technology (10% shrink in this instance). However, the resulting invention is clearly applicable for other technologies. Likewise, the experiments were carried out using reticles taped out to produce SRAM cells. However, the invention is applicable to other memory cells, and other products too. For instance it has also been used to produce circuits with working QESRAM and LOGIC structures in them.

From an imaging perspective, a few things were predetermined:
1. Due to the way the shrinking is achieved (all levels 10% shrink, then re-size the Gate layer back to 180 nm) it was apparent that the LIL holes would have to be imaged as near as possible to the design rule (220 nm) to avoid losing the overlay margins between the Tungsten LILs and their neighbouring Gate (Polysilicon) regions of the ultimately fabricated wafer and between the Active regions and their neighbouring Gate regions of the ultimately fabricated wafer.
2. Imaging contact holes of 220-230 nm dictated that the reticle would have to be a HT-PSM.
3. A side effect of using this HT-PSM reticle would be the appearance of side-lobes. The behaviour of the side-lobes emanating from the slits was not known, but from first principles it was assumed that more tone-inverted light would be present, which would lead to larger side-lobes.
4. The slits would respond differently from the holes, and would require some reticle level manipulations (in length and width) to image them at the correct size.

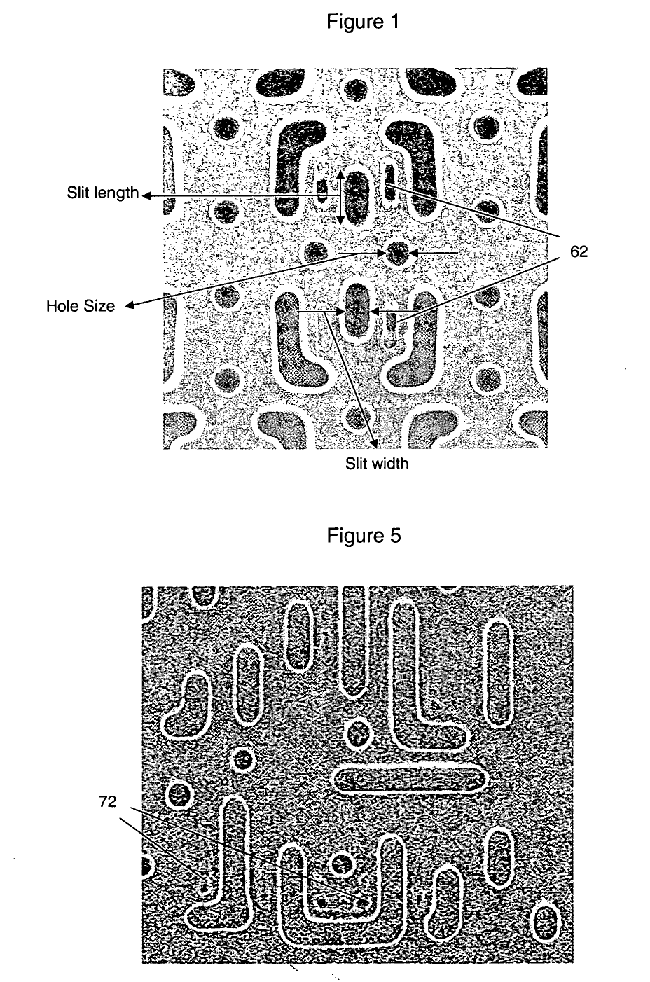

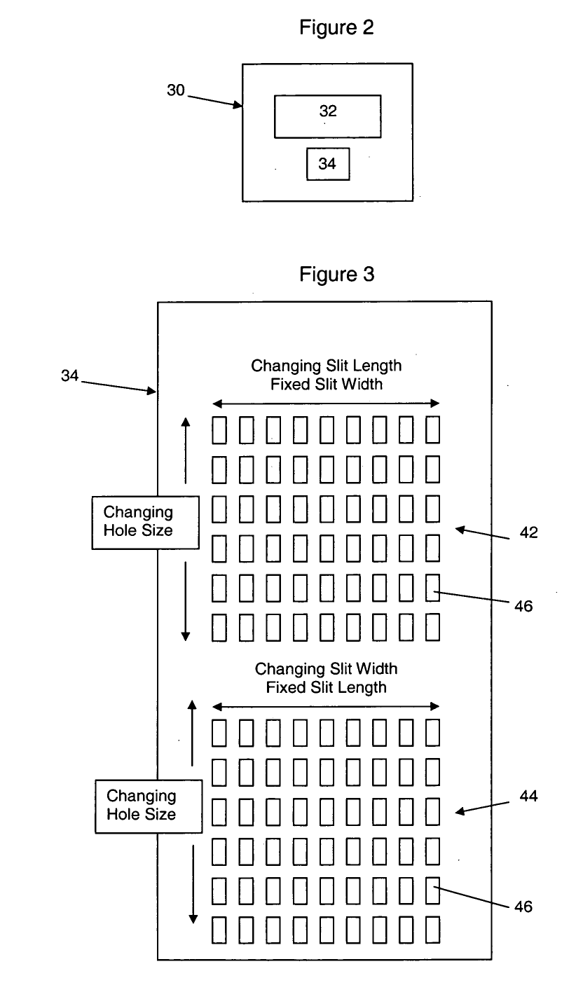

In each experiment a different reticle was taped out, with the form generally shown in FIG. 2. The reticle 30 has two distinct parts, a test chip portion 32 and a LIL test matrix 34. The test chip portion 32 contains the LIL features of interest, varied according to the particular design considerations of the experiment. The LIL test matrix 34 is shown in more detail in FIG. 3. The matrix consists of two arrays 42, 44. Each array consisted of a number of separate and separated test cells 46, in rows and columns. Each row contained nine test cells 46 and each column contained six test cells 46 (thereby providing fifty-four forms per array). Each test cell 46 contained twenty SRAM cells. In the first array 42, the slit width was fixed, whilst the slit length changed, increasing from one column to the next and the hole size also increased from one row to the next. On the other hand, in the second array 44, the slit length was fixed, whilst the slit width changed, increasing from one column to the next and again the hole size increased from one row to the next. What is meant by slit length, slit width and hole size is shown in FIG. 1. Although the test chip portion 32 contains the LIL features of interest, the LIL test matrix 34 was useful in determining how much to adjust the reticle by to achieve the correct on wafer critical dimensions (CD). This relationship was not 1:1. Thus the matrix helped in understanding the non-linear behaviour.

To begin to understand the behaviour of the slits and holes during exposure a first HT-PSM test reticle, Reticle A, was designed and taped out. Following the results of using Reticle A, the design was altered to produce further experimental reticles, Reticle B, then Reticle C.

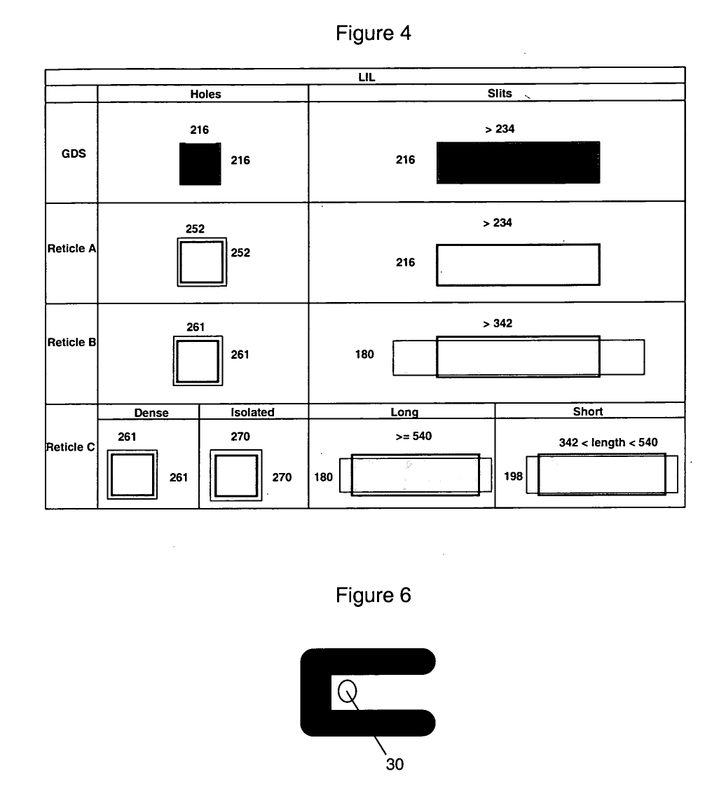

The sizes used for both holes and slits for the LIL in the test chip portion of each experiment are shown in FIG. 4. The first row shows GDS (design data) sizes. Thus the holes are square, at 216 nm by 216 nm and the slits are 216 nm wide by more than 234 nm long. 234 nm is the minimum length for a slit as the smallest step size is 18 nm and anything smaller would therefore be a hole. For Reticles A, B and C, the shaded areas for both holes and slits indicates the size of the features in the reticle, whilst the bold outline shows the relative desired exposed area size.

In a LIL all shapes are made up of holes and slits. Where complex shapes are formed, they can be treated in a linear manner. As long as a slit is of a length greater than the length of the shortest possible slit, it will behave as a slit. With the technology used in the present embodiments, there is a design limitation which restricts the designer from making a very short slit. The shortest slit that the technology standard allows is 234 nm, whilst the hole size is 216 nm. Nothing between 216 and 234 nm is allowed. So all features are either holes or slits.

Reticle A

The sizes used in Reticle A are shown in the second row of FIG. 4. In Reticle A, the LIL features in the test chip portion 12 had first pass sizings; the holes had a size of 252 nm and the slits were left unchanged from the GDS sizings, that is 216 nm wide by more than 234 nm long.

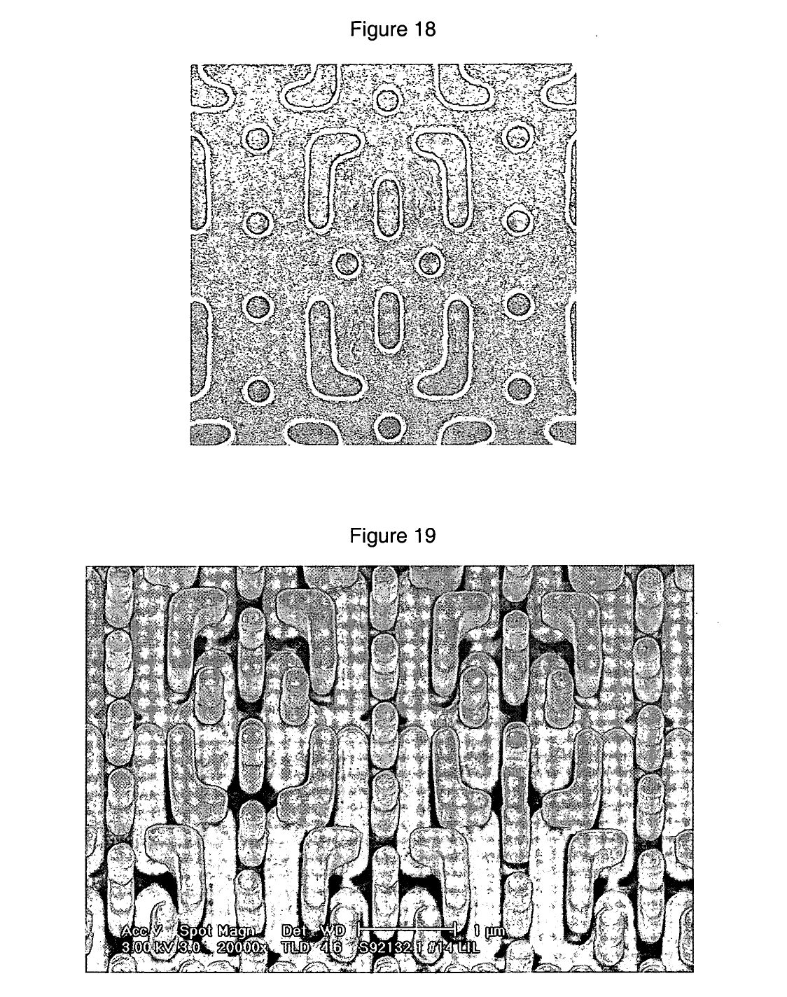

The results from using Reticle A are shown in FIGS. 1 and 5 using a nominal exposure dose (65mj/cM² in this instance), being defined as the correct exposure dose to have the holes patterned at the correct size, and determined by testing. FIG. 1 shows a top scanning electron microscope view of an SRAM after local interconnect layer photolithography using Reticle A. Side-lobing 62 is clearly visible. FIG. 5 shows a top scanning electron microscope view of a controller area of a SRAM after local interconnect layer photolithography. Side-lobing 72 is again clearly visible and the slits are too wide (as compared with the slits of FIG. 18 which are more representative of what is ultimately wanted).

Findings From Reticle A

1. The 252 nm hole was too small. To print a hole of 230 nm required a high exposure dose (around 65mj/cm²), which lead to side-lobing. Although the final CD is intended to be 220 nm (rounded up from 216 nm), there is a −10 nm etch bias in the etching process, which means a printed hole of 230 nm is what is wanted.
2. As suspected, the slits produced serious side-lobes. By using a suitable inspection system and inspecting a step exposure wafer after etching and TiN deposit, the side-lobe “weak point” was determined and is shown as reference 30 in FIG. 6. These three-sided shapes appeared quite frequently in the design of the controller, which meant that a lot of side-lobing would be inevitable, unless the problem was dealt with.
3. The 216 nm slits “blew up” quite noticeably and became far too wide (300 nm) and at the same time they experienced severe line end shortening (by up to 100 nm)
4. The relationship between slit length and slit width could not be separated. This was determined from the LIL test matrix 34 part of Reticle A, where, when the slit width was kept constant and only the slit length was changed, not only did the printed slit length change (as expected) but so did the slit width.
   In summary for Reticle A,
i. Holes—Not OK—Too small, no depth of focus
ii. Slits—Not OK—Too wide, also lost length
iii. Side-Lobes—Not OK—Best energy of 65mj/cm² too close to side-lobing region
   Reticle B

From the sizing matrix present in the Reticle A, the following evolution took place to provide Reticle B. Again, a HT-PSM reticle was taped out, as shown in the third row of FIG. 4, with the following new design rules:
a. The holes were sized up to 261 nm. This would allow the exposure dose to be reduced and move the exposure out of the side-lobing danger region. Side-lobing is related to exposure dose and only occurs when using high exposure doses with a HT-PSM reticle. The side lobing “goes away” when using a lower dose, so the aim was to optimise the hole sizing so it did not need to use a high exposure dose.
b. The slit width was reduced to 180 nm, in an attempt to reduce the “blow up” effect.
c. The slit length was increased by 54 nm per end, in an attempt to counteract the line end shortening.
   Findings From Reticle B

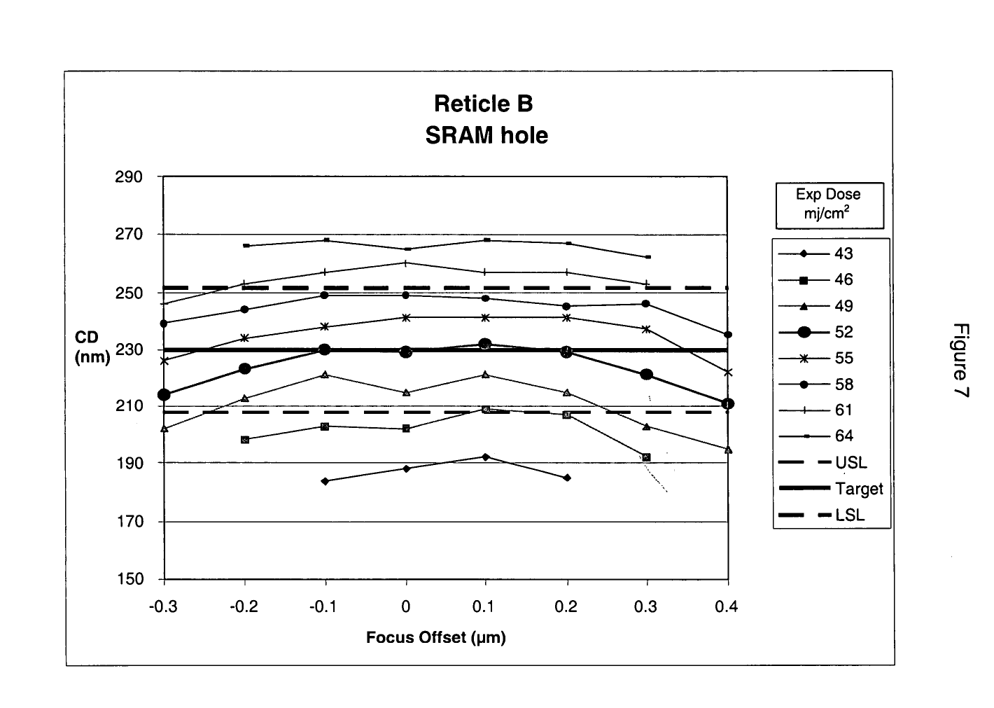

The results for various aspects of Reticle B are shown in FIGS. 7 to 13.

FIG. 7 shows the results of CD (in nm) against focus offset (in μm) for the 261 nm SRAM hole, across a variety of exposure doses, from 43 to 64mj/cm². The target CD was 230 nm, with an upper specification limit (USL) of 253 nm and a lower specification limit (LSL) of 207 nm (around±10% of the CD).
1. The 261 nm SRAM hole responded well, staying between the USL and LSL for a Depth of Focus of 0.7 μm (microns).

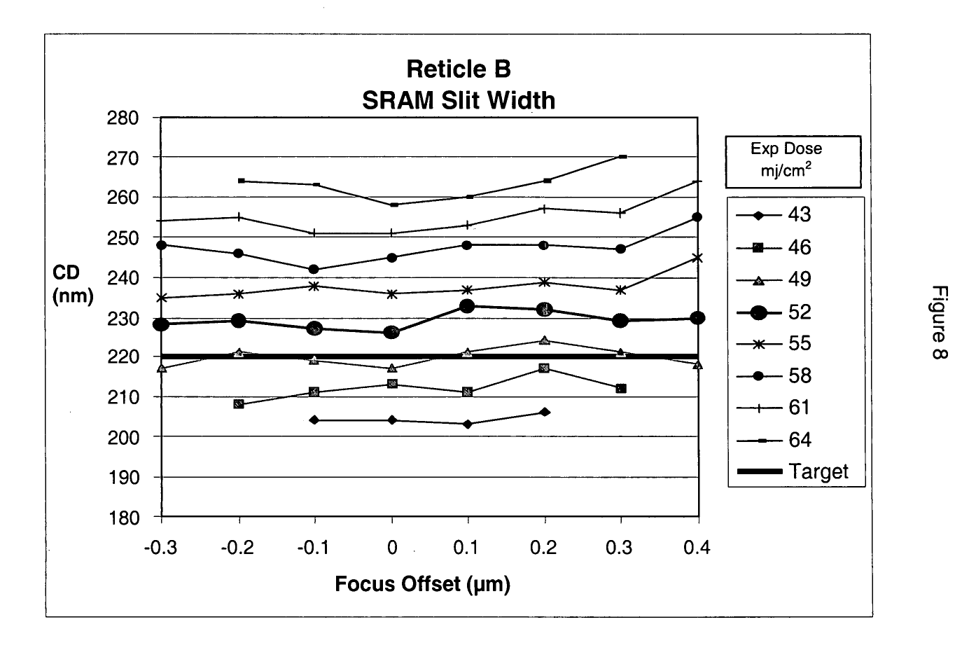

FIG. 8 shows the results of CD (in nm) against focus offset (in μm [microns]) for the 180 nm slit width, across a variety of exposure doses, from 43 to 64mj/cm². The target CD was 218 nm.
2. The SRAM slits performed well, in terms of width, returning almost to their ideal design rule size. These features were also not sensitive to focus.

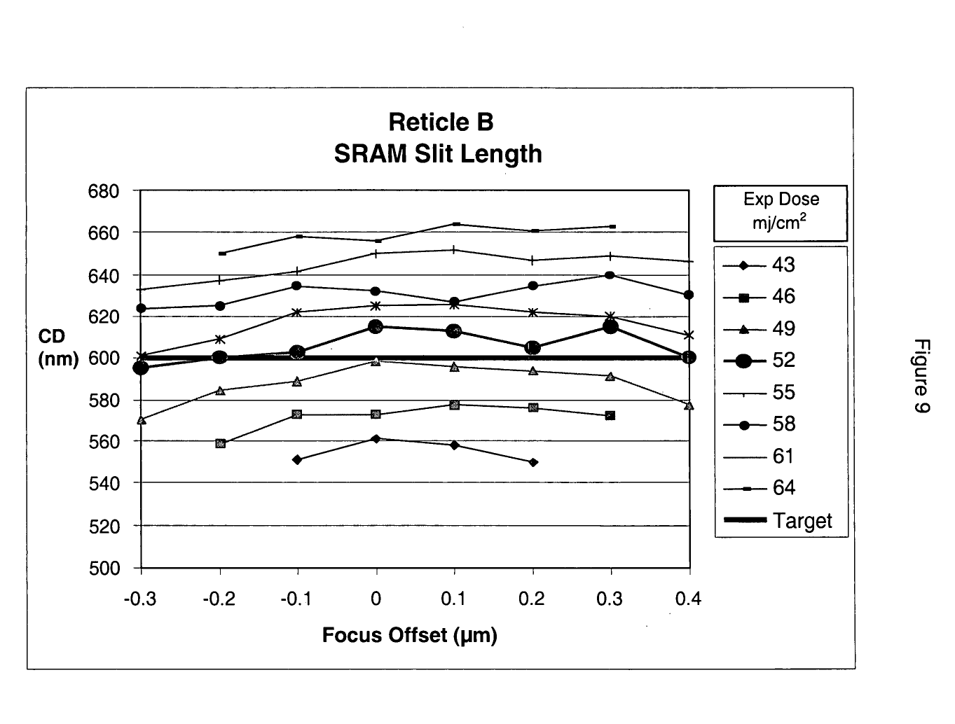

FIG. 9 shows the results of CD (in nm) against focus offset (in μm) for a 708 nm slit length, across a variety of exposure doses, from 43 to 64mj/cm². The target CD was 600 nm.
3. The SRAM slits also performed well, in terms of length, again returning to their ideal design rule size.

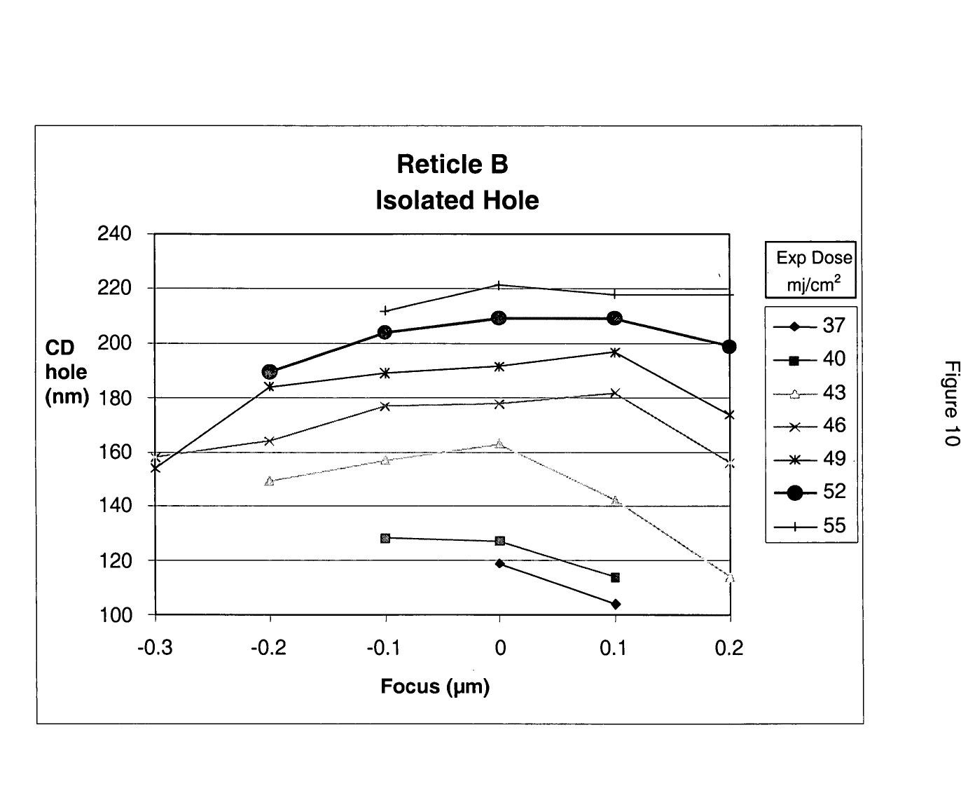

Although in result 1 above, it was stated that the results for the holes were good, further process checks (done as part of a characterisation study) showed there was a significant variation in results. It was determined by the inventors that the variation depended on whether the holes were in a densely packed area, or if they were relatively isolated. FIG. 10 shows the results of CD (in nm) against focus offset (in μm) for an isolated 261 nm SRAM hole, across a variety of exposure doses, from 37 to 55mj/cm².
4. When using the optimum exposure dose to have the SRAM holes correctly patterned at 230 nm, that is 52mj/cm² (optimum based upon the results shown in FIG. 7), the isolated holes produced had a size of only ˜200 nm, with a relatively small depth of focus (0.4 μm (microns)). Such small isolated holes would be difficult to pattern repeatably, and even then would prove very troublesome to etch.

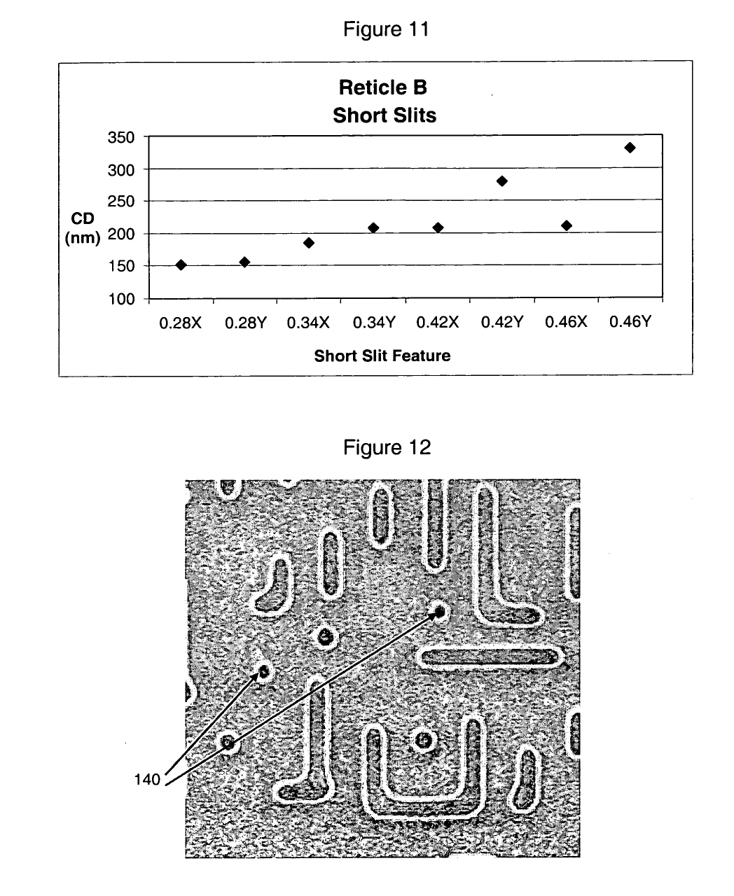

The width of a LIL slit is always 216 nm according to the 180 nm CMOS Shrink design requirements and the length must be greater than 216 nm. Even so, this allows some quite short slits, for instance as appeared in the design in the controller area. These short slits had undergone the same manipulations as the long slits. While the manipulation result for the long slits (e.g. in the SRAM) was very satisfactory, the newly found short slits responded badly to the slit re-sizings carried out in Reticle B. FIG. 11 is a graph showing the lengths and widths of slit features produced given the reticle slit length (to obtain the actual sizes in μm, the slit sizes indicated need to be multiplied by 0.9).
5. From the graph it can be seen that the smallest slit feature produced (from the reticle slit size of 0.28) was only 150 nm wide and 150 nm long. This was clearly outside the processing window, both for photolithography and for etching. The slit was also not behaving as a slit; instead it was behaving like a hole. As the slit length increases at design level, it can be seen that the slit starts to get longer on silicon also, but it is not until the size 0.42 slit that the right width (X direction) is achieved.

Examples of very small slits behaving as holes are shown in FIG. 12, especially two short slits 140.

In summary for Reticle B,
i. Dense holes—OK—Good Depth of Focus (≧0.6 μm) and Exposure Latitude (≧15%) while patterning at 230 nm
ii. Long Slits—OK—Good Depth of Focus and Exposure Latitude while patterning at close to design size at optimum dose
iii. Side-lobes—OK—52 mj/cm² best energy giving good side-lobing margin
iv. Isolated holes—Not OK—Too small
v. Short Slits—Not OK—Too small

Further work was required to correct the isolated holes and the short slits. It was clear the isolated holes needed to be made larger on the reticle, but two questions needed to be answered. How much to size up the holes by, and where was the cut off point when a hole would be classed as isolated? By the same argument the short slits needed similar work and answers, i.e. how much to increase the slit width by, and where was the cut off point when the slits would be classed as short? All of these questions were answered before a new reticle was taped out.

Isolated/Dense Hole Definition

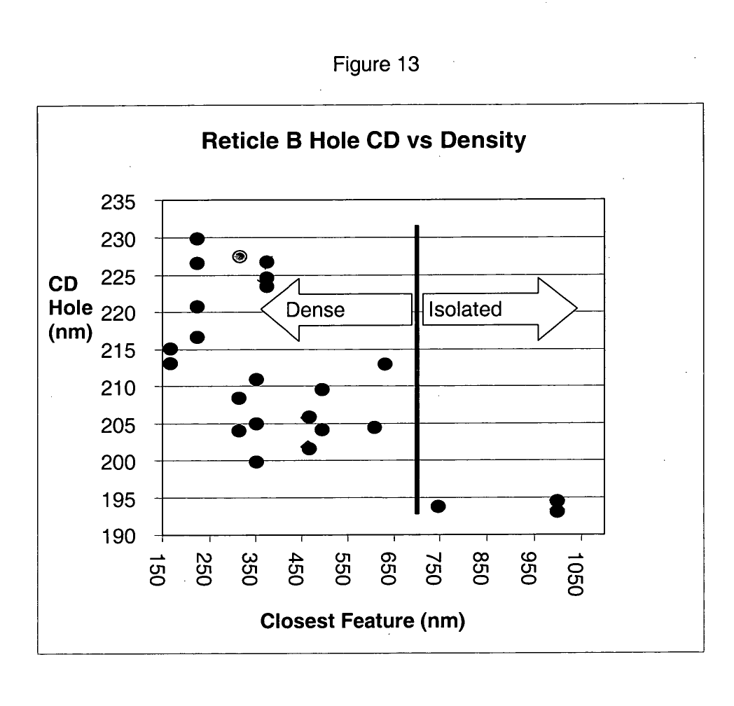

Exposure results from using Reticle B were used to gather data to define isolated and dense (although other structures, for instance a test structure with different contact hole densities could be used). Holes of varying densities were first found, then measured. FIG. 13 is a graph that was generated with the results (CD hole size against distance to the closest feature—edge to edge). The sizes of the holes at design data level were all the same, 261 nm in this instance, but printed on the wafer at different sizes.

From the graph there is a clear relationship between the hole size and its density (i.e. proximity of nearest feature). As the density reduces, the hole gets smaller. From the results of this graph it was decided to define an “isolated hole” as any hole that was further than 680 nm away from the nearest feature, in any direction, allowing the nearest feature to be either another hole, or a slit. The slits themselves did not appear to experience the isolated/dense behaviour exhibited by the holes.

This particular definition may be difficult to implement with some chip finishing scripts used to provide a photomask design. So, for practical purposes this number can be compromised.

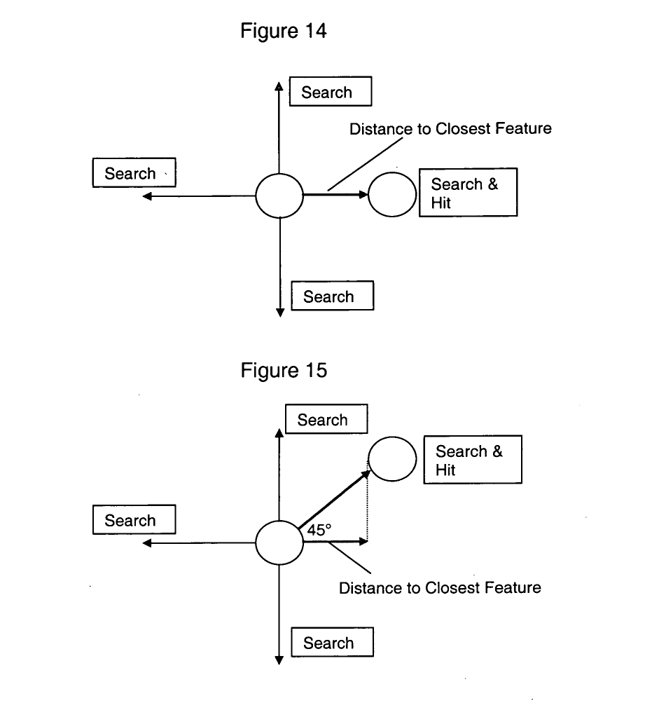

Chip finishing scripts define the nearest feature by looking along both the horizontal axis, and the vertical axis for each hole. Such an approach is shown in FIG. 14. A search is conducted in each orthogonal direction. If there is a hit, then that distance is the distance to the closest feature. If there is- more than one hit, the smallest distance to a hit is the one that counts.

If the nearest feature in the design is not on the horizontal or vertical plane, but for example at 45 degrees, as shown in FIG. 15, then the search still finds it, but the script only returns the distance along one of the two main axes as the distance to the closest feature. The distance returned is the further of the two distances, one along each main axis.

In the worst case, where the closest feature is at 45 degrees and is just far enough away to be counted as an isolated hole, e.g. 681 nm away, the chip finishing script would not give the correct definition of the hole. According to Pythagoras' Theory, the distance along the main axis that would be returned from the script would be 482 nm. So this hole would appear to be dense (since 482 nm, to the nearest feature, is less than 680 nm, the cut off point). Indeed anything at 45 degrees and less than 961 nm would appear dense by this reckoning, which is unacceptable.

As a result of this, the number for defining the cut off between isolated and dense was revised to 450 nm. (480 nm from the Pythagoras above+30 nm for safety margin). This compromise would mean that truly dense holes in the region between 450 nm and 680 nm would be increased in size, when, ideally they would not need to be. However, it was considered better to be sure to correct all isolated holes by also over-correcting a few which were not technically isolated.

Where a chip finishing script is able to measure distances in directions other than just along the two main axes, the measurement and definition of isolated vs. dense can be further and more accurately defined.

The sizing required for these isolated holes had been calculated previously from the LIL test matrix in Reticle A and was defined as 270 nm (i.e. an upsize of 9 nm compared to a dense hole—this was controlled by the reticle grid size of 9 nm in this case, an upsizing of 18 nm would have been too much, as the LIL test matrix in Reticle A showed).

Short and Long Slit Definition

The short and long decision was extrapolated from the already existing graph of the small slits in the controller, shown in FIG. 11

From the graph it was decided that most of these slits were too small, except perhaps the size 0.46 slit, which would be acceptable as it was, or could equally survive if it was made bigger. So the next size of slit, 0.48 would clearly be acceptable as it was. This size 0.48 slit was chosen as the cut off between short and long. To get the actual number to be used in the chip finishing script, the following calculations were done:

At 180 nm CMOS GDS level the 0.48 slit is 480 nm long.

For 180 nm CMOS shrink 10%, the slit becomes 0.48×0.9=432 nm long.

As the decision on whether a slit is short or long slit is made in the chip finishing script after the line end extensions have been globally added, the relevant slit length becomes 432+54+54=540 nm (see the definition “c” of Reticle B for the added 54 nm at each end).

So the cut off in the chip finishing script becomes 540 nm. Any slit shorter than this will be widened. Any slit longer than this will not be manipulated further.

Short Slit Manipulation

The width of the newly defined short slits was extrapolated as follows:

180 nm, the current setting on Reticle B was too small. 216 nm, the original setting on Reticle A was too big. Hence 198 nm was the width chosen for the short slits. Due to finite gridsize used in chip finishing manipulation, only discrete interval steps of 18 nm could be chosen for the slits (although for the holes, 9 nm grid steps could be achieved).

Reticle C

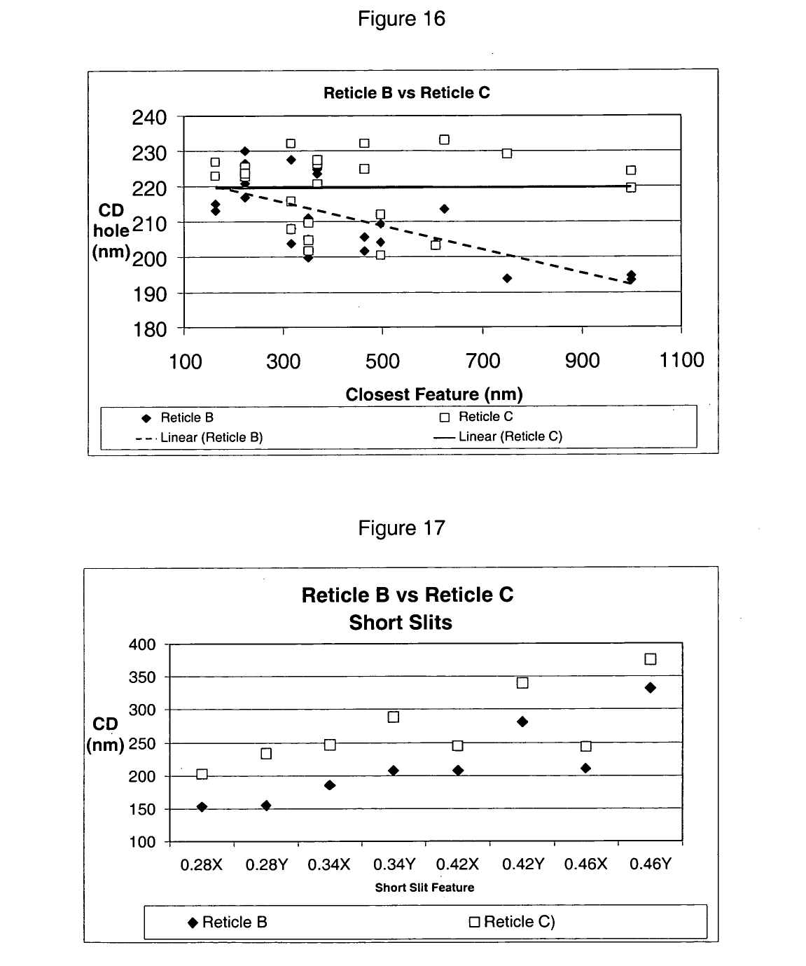

Combining the improvements made on Reticle B, and adjusting now for isolated holes, and for short slits, the following evolution took place on Reticle C. Again a HT-PSM reticle was taped out, as shown in the fourth row of FIG. 4, with the following new design rules:
a. All holes were checked for density. If the distance to the nearest feature (hole or slit) was greater than 450 nm, the hole was deemed as an isolated hole and was sized up to 0.270 μm. All other holes were deemed as dense holes and remained the same size at 0.261 μm.
b. All slits were checked for length. If the length (after 10% shrinking+line end extending) was less than 0.54 μm, this feature was deemed as a short slit and the slit width was set to 0.198 μm. All other slits were deemed as long slits and the width remained the same as before at 0.18 μm
c. The same line end extensions of 54 nm per end were applied as with Reticle B.
   Findings From Reticle C
1. The good dense hole performance was unchanged from Reticle B
2. The isolated holes had increased in size, to give almost zero isolation vs. dense bias, as shown in FIG. 16. This graph shows CD hole size against actual distance to closest feature, for both Reticles B and C (thus it includes the data of FIG. 13). Approximate linear trend lines are included, showing the size for holes in Reticle C was constant with distance, whilst it decreased with distance for Reticle B. It will be recalled, from above, that holes with the nearest feature between 450 nm to 680 nm away were in danger of being oversized unnecessarily (due to the limitations in determining distances for features not along the major axes). It can be seen that FIG. 16, bears this out, in that the larger holes tended to be those with the nearest features from 450 nm to 680 nm away. However, the effect of the oversizing of these holes was negiglible.
3. The short slits increased in size, as is shown in FIG. 17. This graph shows CD slit size against Reticle slit size, for both Reticles B and C (thus it includes the data of FIG. 11). The CD width for most slits went up to around 250 nm, which is larger than the desired width of 216 nm. However, it is better that they be too large rather than<200 nm, which is the smallest dimension that typically can be etched open.

In summary for Reticle C,
i. Dense holes—OK—Good Depth of Focus and Exposure Latitude while patterning at 0.23 μm
ii. Long Slits—OK—Good Depth of Focus and Exposure Latitude while patterning at close to design size at optimum dose
iii. Side-lobes—OK—52mj/cm² best energy giving good side-lobing margin
iv. Isolated holes—OK—almost no isolation vs. dense bias seen
v. Short Slits—OK—patterned at close to design size

Although there were problems with Reticle A and Reticle B as described earlier, both could be used in their own operating regions. Reticle A could be used at a hole CD of around 0.24 μm, at which time the slits were very wide. Products produced from this reticle showed a tendency to leak electrically as a result of these wide slits. Reticle B had to be overexposed considerably to around 260-270 nm to overcome the problems of the isolated holes and short slits. These problems were successfully overcome with Reticle C.

FIG. 18 is a top scanning electron microscope view of an SRAM after local interconnect layer photolithography, produced using reticle 3. As such it is an acceptable result, showing that the present invention is successful.

FIG. 19 is a tilted scanning electron microscope view of the same SRAM structure after “reverse-engineering”, with the Tungsten filled local interconnect layer contacts and lines clearly arising from the silicon surface.

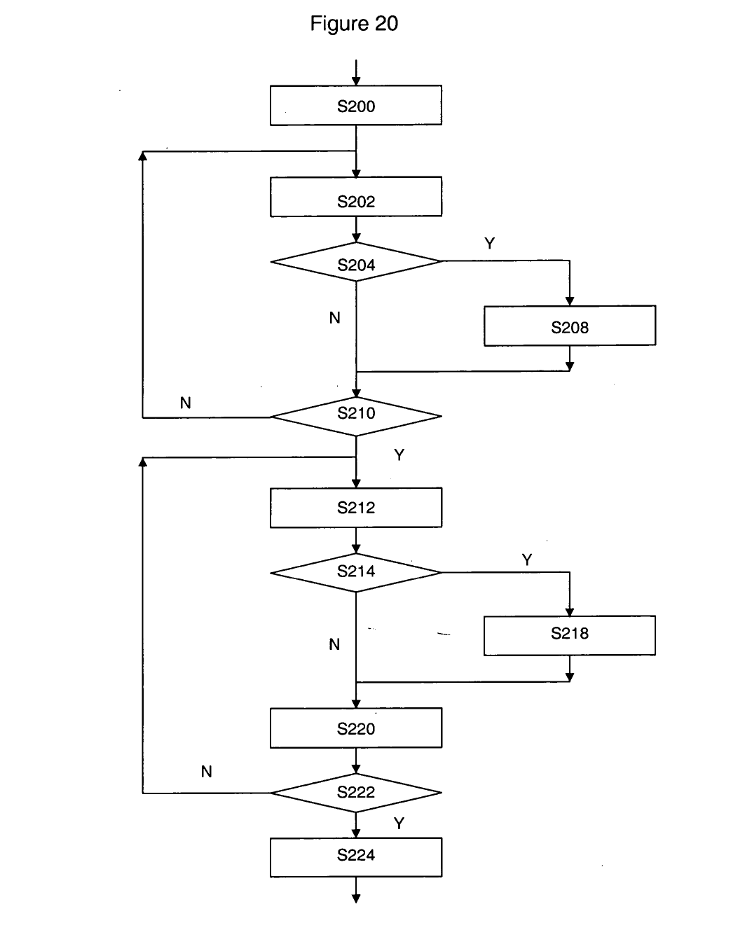

Thus for future reticle design a series of steps can be followed as shown in the flowchart of FIG. 20.

In step S200, design data is input and holes and slits etc. for the reticle are determined accordingly. Step S202 causes a hole to be found. Step S204 determines whether the hole is defined as isolated, according to the prevalent definition (in the above embodiment that is if the nearest feature is at more than 450 nm away [along a major axis]). If it is isolated, then in step S208 the hole is sized up from a first size (in the above embodiment that is 261 nm) to a second size (in the above embodiment that is 270 nm), otherwise it remains at the first size. In both instances, the next step is S210.

If some holes have not yet been checked, then step S210 returns the run to step S202 to look for another hole to be checked. However, once all the relevant holes have been checked, the run proceeds to step S212. Step S212 causes a slit to be found. Step S214 determines whether the slit is defined as small, according to the prevalent definition (in the above embodiment that is if following 10% shrinkage and 54 nm extension per end, the slit length would be less than 540 nm). If it is small, then in step S218 the slit width is sized up from a first width (in the above embodiment that is 180 nm) to a second width (in the above embodiment that is 198 nm), otherwise it remains at the first width. In both instances, the next step is S220, where the slits are extended a first slit length extension amount per end (in the above embodiment that is 54 nm).

Step S222 follows. If some slits have not yet been checked, then step S222 returns the run to step S212 to look for another slit to be checked. However, once all the relevant slits have been checked, the run proceeds to step S224, where the final design (or at least final for this part of the process) is output.

In the above flowchart, the step S220 of extending the ends of the slits occurs after the widths have been manipulated. This can happen before, for instance as part of the initial step S200 or between steps S212 and S214 (this would of course alter the definition of “small slit” in step S214). Other portions of the run can be in different orders too, for instance the slits could be dealt with before the holes. An alternative is to search through each feature and deal with it according to whether it is a hole or slit and isolated or small, respectively, before looking for the next feature. There are many other possibilities.

The results of the investigations carried out as described above, indicate only two results at a time (isolated vs. dense, short slit vs. long slit). According to the circumstances, it may be useful to split each feature up into three or more categories of this ilk or others (e.g. isolated, non-isolated and dense, or short, medium and long, etc.).

The solutions proposed in the present instance with the current chip finishing scripts may not be ideal, but have the benefit of practicality, without the need for a solution provided by full model based Optimal Proximity Correction. This may provide good results, but it tends to be more complex and expensive than is justified, especially given the results achievable with the present invention.

In the above description many measurements appear accurate to the nearest nm. This is not necessarily supposed to imply accuracy to that level. In many cases it refers to sizes within an industry standard or technology node.

The manipulations on Reticle C answered all of the problems encountered during the development of a single reticle for 180 nm CMOS Shrink. Any new CMOS Shrink product can use the same manipulations as Reticle C. Likewise, non-shrink 180 nm CMOS LIL is known to have isolation vs. dense bias and, although not as severe as the shrink process, it still reduces the photo and etching process window. It is possible, using the knowledge gained from this 10% shrink, to manipulate the non-shrink LIL reticle to design a test reticle to obtain the correct manipulations to remove this bias effect effectively.

1. A method for use in designing a reticle for exposing a substrate during production of a circuit, comprising:
determining the relative isolation of first type features to be produced using the reticle with respect to adjacent features;
sizing first type apertures in the reticle corresponding to the first type features using different sizing rules depending on the relative isolation of the first type features;
determining dimensions of second type features to be produced using the same reticle; and
sizing second type apertures corresponding to the second type features in the same reticle using different sizing rules depending on the dimensions of the second type features.

2. A method according to claim 1, wherein the results of the relative isolation determining step cause the more isolated first type apertures to be made larger.

3. A method according to claim 1, wherein the relative isolation is determined against a first threshold, with the more isolated first type apertures being sized a first way and the less isolated first type apertures sized a second way.

4. A method according to claim 1, further comprising the step of extending the size of at least some of the second type apertures in a second dimension orthogonal to a first dimension.

5. A method according to claim 1, wherein the results of the first dimension determining step cause the second dimension of the smaller second type apertures to be made larger.

6. A method according to claim 1, wherein the first type features comprises a plurality of holes.

7. A method according to claim 1, wherein the second type features comprises a plurality of slits.

8. A method according to claim 1, wherein the results of the first dimension determining step cause the second type apertures to be sized in the second dimension, orthogonal to the first dimension.

9. A method according to claim 1, wherein the second type apertures corresponding to the second type features with the smaller first dimension are increased in the second dimension more than those with the larger first dimension.

10. A method according to claim 1, wherein the first dimension is compared with a second threshold, with the larger second type apertures being sized a third way and the smaller second type apertures sized a fourth way.

11. A method according to claim 1, wherein the first dimension of a feature is the length of the feature.

12. A method according to claim 1, wherein the features are features of memory cells.

13. A method according to claim 1, wherein the reticle is a Half-Tone Phase Shift Mask.

14. A method according to claim 1 for producing features for a local interconnect layer.

15. A reticle for exposing a substrate during production of a circuit, comprising:
a plurality of first type apertures sized using different sizing rules depending on the relative isolation of first type features of the circuit corresponding to the first type apertures; and
a plurality of second type apertures sized using different sizing rules depending on the dimensions of second type features of the circuit corresponding to the second type apertures.

16. A reticle according to claim 15, wherein the more isolated first type apertures are relatively larger than the less isolated first type apertures corresponding to the size of features to be produced.

17. A reticle according to claim 15, wherein the first type apertures are apertures for producing holes.

18. A reticle according to claim 15, wherein the second type apertures are apertures for producing slits.

19. A reticle according to claim 15, wherein the size in a first dimension determines size in a second dimension, orthogonal to the first dimension.

20. A reticle according to claim 15, wherein the second type features determined to be small in the first dimension are larger in the second dimension than those determined to be the larger in the first dimension.

21. A reticle according to claim 15, wherein larger and smaller in the first dimension is determined by comparison with a threshold.

22. A reticle according to claim 15, wherein the first dimension of a feature is the length of the feature.

23. A reticle according to claim 15, wherein the features are features of memory cells.

24. A reticle according to claim 15, wherein the reticle is a Half-Tone Phase Shift Mask.

25. A reticle according to claim 15, being a local interconnect layer reticle.

26. A reticle designed according to the method of claim 1.

27. A reticle for exposing a substrate during production of a circuit, comprising:
a plurality of first type apertures sized using different sizing rules depending on the relative isolation of first type features of the circuit corresponding to the first type apertures; and
a plurality of second type apertures sized using different sizing rules depending on the dimensions of second type features of the circuit corresponding to the second type apertures, designed according to the method of claim 1.

28. A method of producing an integrated circuit comprising the steps of:
designing a reticle in accordance with the method of claim 14;
producing the so designed reticle; and
using said reticle to expose features of both the first and second types in a single step in at least one layer of at least a portion of the integrated circuit.

29. A method of producing an integrated circuit comprising using a reticle as defined in claim 15 to print features of both the first and second types in a single step in at least a portion of the integrated circuit.

30. An integrated circuit, at least a portion of which is produced using a reticle as defined in claim 15.
